(19) United States
(12) Patent Application Publication
(10) Pub. No.: US 2025/0320451 A1
(43) Pub. Date: Oct. 16, 2025

(54) STRAIN OF ACID-TOLERANT CYBERLINDNERA JADINII AND ITS APPLICATION

(71) Applicants: Chifeng Ruiyang Chemical Co. Ltd., Chifeng (CN); Inner Mongolia University of Technology, Hohhot (CN)

(72) Inventors: Hongdong Quan, Chifeng (CN); Xiuguo Yang, Chifeng (CN); Hongyan Li, Chifeng (CN); Luqian Ye, Chifeng (CN); Xiaohe Li, Chifeng (CN); Zhanying Liu, Hohhot (CN); Yingnan Chai, Hohhot (CN); Xinyao Xie, Hohhot (CN)

(21) Appl. No.: 18/988,847

(22) Filed: Dec. 19, 2024

Related U.S. Application Data
(63) Continuation of application No. PCT/CN2024/131280, filed on Nov. 11, 2024.

(30) Foreign Application Priority Data
Apr. 11, 2024 (CN) 202410434830.9

(51) Int. Cl. C12N 1/16; C12R 1/645

(52) U.S. Cl. CPC C12N 1/165; C12R 2001/645

(57) ABSTRACT

This application discloses an acid-tolerant Cyberlindnera jadinii yeast strain and its applications, belonging to the field of microbial technology. The Cyberlindnera jadinii yeast strain is Cyberlindnera jadinii C-12-2, which was deposited on Mar. 5, 2024, at the Guangdong Microbial Culture Collection Center, with the deposit number GDMCC NO: 64388. The Cyberlindnera jadinii yeast disclosed in this application can grow in a medium with a pH of 3.5, and its crude protein content can reach 70.02% after 24 hours of fermentation. The yeast strain disclosed in this application was obtained through heavy-ion irradiation mutagenesis followed by acid adaptation. It has been determined that the strain can grow in an acidic medium with a pH of 3.5, overcoming the growth limitation of the original strain at low pH values.

### CROSS-REFERENCE TO RELATED APPLICATIONS

This application is a continuation application of PCT Patent Application No. PCT/CN2024/131280, filed on Nov. 11, 2024, which claims priority to Chinese Patent Application No. 202410434830.9, filed on Apr. 11, 2024, the contents of which are incorporated herein by reference in their entirety.

### TECHNICAL FIELD

This application belongs to the field of microbial technology, specifically relates to a strain of acid-tolerant Cyberlindnera jadinii and its application.

### BACKGROUND

Currently, the annual import of protein feed in China exceeds 100 million tons, and the shortage of protein feed severely impacts the development of the livestock and animal husbandry sectors. Therefore, more and broader protein feed resources are needed in livestock production. Among these, single-cell protein, which has a high protein content, rich nutrients, and is not affected by seasonal and climatic conditions, is an important way to address the shortage of protein feed resources and achieve resource substitution. Candida utilis, as one of the excellent strains for high-yield microbial fermented feed protein, is rich in vitamin B and protein. It plays a role in regulating the intestinal microecological balance of animals, improving feed digestibility, and enhancing animal immunity. It also has advantages such as a short cultivation cycle, good safety, and excellent fermentation performance. It can utilize industrial by-products such as molasses, starch factory wastewater, and brewers' spent grains as carbon sources, while using urea or nitrate as nitrogen sources for growth in fermented feed. However, the acid tolerance of existing strains is relatively low, so there is a need to cultivate strains with higher acid tolerance.

### SUMMARY

The purpose of this application is providing a strain of acid-tolerant Cyberlindnera jadinii and its application, in order to solving the technical issue of relatively low acid resistance in existing Candida utilis strains.

To achieve the above objective, the technical solution adopted by this application is as follows:

A strain of acid-tolerant Cyberlindnera jadinii, the Cyberlindnera jadinii is Cyberlindnera jadinii C-12-2, which was deposited on Mar. 5, 2024, at the Guangdong Microbial Culture Collection Center with the deposit number GDMCC NO: 64388. The deposit address is: 5th Floor, Building 59, No. 100 Xianlie Central road, Guangzhou, Guangdong Province, China.

The Cyberlindnera jadinii C-12-2 strain provided by this application is obtained through heavy ion radiation mutagenesis followed by acid-resistant domestication, and the strain can grow in an acidic medium with a pH of 3.5, and after 24 hours of fermentation, the crude protein content can reach 70.02%. It can overcome the obstacle of the original strain being unable to grow at low pH levels. Stability tests show that after 12 consecutive subcultures, the single-cell crude protein yield of this mutant strain did not fluctuate significantly, proving that it has stable genetic traits. This strain exhibits higher acid resistance and protein production capabilities, providing a high-quality strain resource for mixed-culture fermentation production of microbial protein, microbial protein feed, or yeast cultures.

Based on this strain, the invention also provides the application of this acid-resistant Cyberlindnera jadinii as a fermentation strain in the preparation of microbial protein through single or mixed-culture fermentation, or as a raw material in the production of yeast cultures.

This application has the following beneficial effects:

Cyberlindnera jadinii is a sexual or asexual form of Candida utilis. The present application provides an acid-resistant, high-yielding biomass protein strain of Cyberlindnera jadinii (C-12-2). This strain can grow in a medium with a pH of 3.5, and after 24 hours of fermentation, the crude protein content can reach 70.02%. The Cyberlindnera jadinii strain described in this application was obtained through heavy ion irradiation mutation followed by acid resistance acclimatization. It has been determined that this strain can grow in an acidic medium with a pH of 3.5, and can overcome the obstacle which original strain can not grow at low pH. It is a more acid-resistant strain with higher protein production ability and it can provide a high-quality resource for mixed fermentation to produce biomass protein, protein feed, or yeast cultures.

### BRIEF DESCRIPTION OF THE DRAWINGS

FIG. 1 is the strain acclimatization flowchart;

FIG. 2 is the growth curve of the Cyberlindnera jadinii C-12-2 strain;

FIG. 3 is a comparison of the crude protein content in the biomass of Pichia jadinii C-0, C-12, and Cyberlindnera jadinii C-12-2;

FIG. 4 shows the experimental results for the stability of the biomass protein yield performance of Cyberlindnera jadinii C-12-2.

### DETAILED DESCRIPTION OF THE EMBODIMENTS

The following further illustrates the present invention in conjunction with various embodiments and accompanying drawings. The methods of the present application include, but are not limited to, the following embodiments.

### First Embodiment

This embodiment utilizes the Lanzhou Heavy Ion Research Facility (HIRFL) at the Institute of Modern Physics, Chinese Academy of Sciences, to perform irradiation mutation screening for high-yield microbial protein strains. The specific method is as follows:
1. The Candida utilis (purchased from the China General Microbiological Culture Collection Center, accession number CGMCC 2.2878) was taken out from a −70° C. freezer and allowed to thaw slowly at room temperature. It was then inoculated into 100 mL of YPD liquid medium at a 1% inoculation quantity and placed into a constant-temperature shaker for culturing overnight which the culture conditions were: temperature 30° C., shaking speed 180 rpm, and culture time 24 hours. The OD₆₀₀ was measured to obtain a cell suspension with OD₆₀₀=0.8, which was recorded as the starting inoculum.
2. Take 2 mL of the starting inoculum and place it into sterile irradiation dishes, sealing with sealing film. Then, irradiate with a ¹²C⁶⁺ ion beam at 80 MeV/u, with irradiation doses of 0 Gy, 40 Gy, 80 Gy, 120 Gy, 160 Gy, and 200 Gy, respectively.
3. Dilute the cell suspensions with different irradiation doses to 10-5 to obtain cell suspensions with different irradiation doses. Then, take 50 μL of each cell suspensions with different irradiation doses and spread it onto YPD solid medium and then incubate at 30° C. for 48 hours to obtain the strains after different irradiation doses.
4. The strains after different irradiations were each inoculated into 100 mL of YPD medium and cultured overnight in a constant-temperature shaker. The culture conditions were: temperature 30° C., shaking speed 180 rpm. The OD₆₀₀ was measured to obtain a cell suspension with OD₆₀₀=0.8, which was then used as the activated inoculum.
5. Initial screening of the strains: Strains with acid resistance and high biomass protein production were selected based on colony morphology and growth characteristics.

The specific method was as follows: The activated cell suspensions obtained in step 4 were diluted at gradients of 10⁻³, 10⁻⁴, 10⁻⁵, and 10⁻⁶, and 50 μL of each dilution was spread onto starch selection medium. The plates were then placed in a 30° C. constant-temperature incubator for culture. After the colonies grew, the larger colonies were selected and transferred to YPD liquid medium, where they were cultured at 30° C., 180 rpm for 48 hours. The cell concentration (OD₆₀₀ at a wavelength of 600 nm) was measured to determine the growth rate of the cells. Strains with faster growth rates and higher biomass protein content were selected for further screening.

In this experiment, 21 strains with larger colony diameters than the original strain were screened from 2080 individual colonies. The strains were named according to their irradiation doses, and the liquid culture results are shown in Table 1. These 21 mutant strains were selected for further re-screening.

Table 1 is initial Screening of Colony Diameter and Biomass Concentration (OD₆₀₀) Results

| | number of | | Biomass |
|---|---|---|---|
| | Mutagenized | colony | Concentration |
| irradiation | Candida | diameter | OD₆₀₀ (9-fold |
| dose (Gy) | utilis strain | (mm) | dilution) |
| 0 | C-0 | 5.8 | 0.842 |
| 40 | C-1 | 6 | 0.975 |
| | C-2 | 6.2 | 0.899 |
| | C-3 | 5.9 | 1.062 |
| 80 | C-4 | 6.9 | 0.874 |
| | C-5 | 6.1 | 0.838 |
| | C-6 | 7.1 | 0.865 |
| | C-7 | 6.9 | 0.869 |
| | C-8 | 6.8 | 0.831 |
| 120 | C-9 | 6.1 | 0.848 |
| | C-10 | 6.8 | 0.821 |
| | C-11 | 6.1 | 0.880 |
| | C-12 | 6.7 | 0.800 |
| | C-13 | 7.3 | 0.864 |
| | C-14 | 7.1 | 0.874 |
| | C-15 | 7.8 | 0.829 |
| | C-16 | 7 | 0.848 |
| 160 | C-17 | 6.3 | 0.852 |
| | C-18 | 7.1 | 0.884 |
| 200 | C-19 | 6.1 | 0.955 |
| | C-20 | 7.3 | 0.892 |
| | C-21 | 7 | 0.866 |

6. Re-screening of the strains: The 22 strains listed in Table 1 were each activated, and the specific method is as follows:

The sample was taken out of the −70° C. freezer and allowed to thaw slowly at room temperature. Then, a 1% inoculum (v/v) was added to 100 mL of YPD liquid medium and incubated overnight in a shaking incubator under the conditions of 30° C. and 180 rpm. The OD₆₀₀ was measured to obtain an activated bacterial culture with OD₆₀₀=0.8.

Prepare the culture liquids for 22 strains as follows:

After activating the 22 strains of Saccharomyces cerevisiae, inoculate each strain into YPD liquid medium at a 1% (v/v) inoculum. Incubate at 30° C. and 180 rpm for 48 hours to obtain the culture liquid. Then, measure the microbial biomass and the protein content of the cells, as described below:
(1) Centrifuge the cell suspension at 8000 rpm for 5 minutes, discard the supernatant, and then wash the cells with sterile water by centrifuging at 8000 rpm for 5 minutes. Discard the supernatant and repeat the washing process 3 times. Collect the cells and dry them at 90° C. until constant weight is achieved. The sample weight (dry weight) is the biomass of Saccharomyces cerevisiae.
(2) Determine the crude protein content (% crude protein) in the sample according to the method outlined in GB/T 6432-2018. The crude protein content represents the single-cell protein content. The calculation formula is as follows:

$$Crude protein ( \% ) = \frac{( {V}_{2} - {V}_{1} ) \times C \times 0.014 \times 6.25}{m \times {V}^{′} / V} \times 100$$

In the formula:
V₂—Volume of standard hydrochloric acid solution (mL) consumed by the titrated sample;
V₁—Volume of standard hydrochloric acid solution (mL) required for the blank control;
C—Concentration of the standard hydrochloric acid solution;
m—Mass of the sample (g);
V—Total volume of the sample digestion solution (mL);
V′—Volume of digestion solution for distillation (mL);
0.0140—The grams of nitrogen equivalent per milligram;
6.25—The average conversion factor for nitrogen to protein.

The results for the biomass and crude protein content of the 22 strains are shown in Table 2.

TABLE 2
Biomass of the re-screened colonies and their crude
protein content in the fermentation broth.

| | Mutation-derived | | Crude |
|---|---|---|---|
| | Saccharomyces | | protein |
| irradiation | cerevisiae | Biomass | content |
| dose (Gy) | strain number | (g/L) | % |
| 0 | C-0 | 3.753 | 52.29 |
| 40 | C-1 | 3.980 | 58.90 |
| | C-2 | 2.735 | 50.32 |
| | C-3 | 3.591 | 52.52 |
| 80 | C-4 | 3.550 | 48.90 |
| | C-5 | 3.497 | 50.57 |
| | C-6 | 3.461 | 47.37 |
| | C-7 | 3.005 | 49.34 |
| | C-8 | 3.621 | 49.20 |
| 120 | C-9 | 3.624 | 53.68 |
| | C-10 | 3.469 | 46.03 |
| | C-11 | 3.662 | 48.70 |
| | C-12 | 3.631 | 64.88 |
| | C-13 | 3.500 | 52.32 |
| | C-14 | 3.668 | 53.68 |
| | C-15 | 3.423 | 60.02 |
| | C-16 | 3.624 | 50.57 |
| 160 | C-17 | 3.652 | 49.53 |
| | C-18 | 3.493 | 45.02 |
| 200 | C-19 | 3.526 | 50.25 |
| | C-20 | 3.562 | 50.54 |
| | C-21 | 3.497 | 49.14 |

As shown in Table 2, the crude protein content of the 5 positive-mutant strains exhibited varying degrees of increase compared to the original strain. Notably, when the irradiation dose reached 120 Gy, one positive mutant strain with the highest crude protein content was obtained, which was named Saccharomyces cerevisiae C-12. Its crude protein content reached 64.88%, representing a 24.08% increase compared to the original strain Saccharomyces cerevisiae CGMCC 2.2878, which had a crude protein content of 52.29%.

### Second Embodiment

In this example, the method of gradually lowering the pH of the culture medium was used to acid-tolerant acclimate the Saccharomyces cerevisiae C-12 strain, which had been selected for high cell protein production after heavy ion mutation screening.

The details are as follows:

### 1. Strain Acclimatization

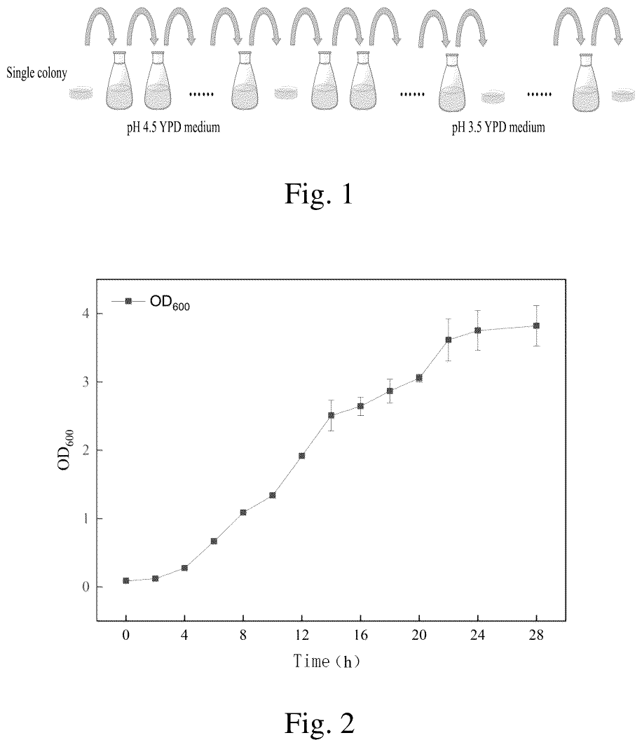

The strain was acclimatized using the method of gradually lowering the pH of the culture medium (as shown in FIG. 1). The high cell protein-producing Saccharomyces cerevisiae C-12 strain culture was inoculated into a fresh YPD liquid medium with a pH of 4.5 at a 1% inoculum and incubated at 30° C. and 180 rpm for 24 hours. Afterward, it was transferred to a fresh YPD liquid medium with the same pH of 4.5, and this process was repeated for one month, with sub-culturing and plate purification performed every week. Based on this procedure, the yeast strain adapted to pH 4.5 was then transferred to a YPD liquid medium with a pH of 3.5 for further acclimatization. Eventually, a low-pH-tolerant, high cell protein-producing Saccharomyces cerevisiae strain was obtained and named Saccharomyces cerevisiae C-12-2.

After adapting the high-yield protein-producing Candida utilis C-12 strain, the acid-tolerant and high-yielding strain Cyberlindnera jadinii C-12-2 was obtained. Observations revealed that the acid-adapted Cyberlindnera jadinii C-12-2 could adapt well to low-pH growth conditions, overcoming the limitation of Candida utilis C-12, which is unable to grow in YPD medium with a pH of 3.5.

The high-yield protein-producing Candida utilis C-12 strain was adapted to obtain the acid-tolerant, high-yield protein-producing strain Cyberlindnera jadinii C-12-2. Observations revealed that the acid-adapted Cyberlindnera jadinii C-12-2 could adapt well to low-pH growth conditions, overcoming the inability of Candida utilis C-12 to grow in YPD medium with a pH of 3.5.

### 2, Growth Curve Measurement

The acclimatized Saccharomyces cerevisiae C-12-2 seed culture was inoculated at a 1% (v/v) inoculum (OD₆₀₀=0.8) into 100 mLL YPD liquid medium, with a blank control group included. The cultures were incubated at 30° C. and 180 rpm with shaking. Samples of 1 mL were taken at 0 h, 2 h, 4 h, 6 h, 8 h, 10 h, 12 h, 14 h, 16 h, 18 h, 20 h, 22 h, 24 h, and 28 h post-inoculation, with the inoculated medium used for baseline calibration. The absorbance at 600 nm (OD600) of the two yeast strains after acclimatization was measured. A growth curve was plotted with absorbance as the vertical axis and growth time as the horizontal axis.

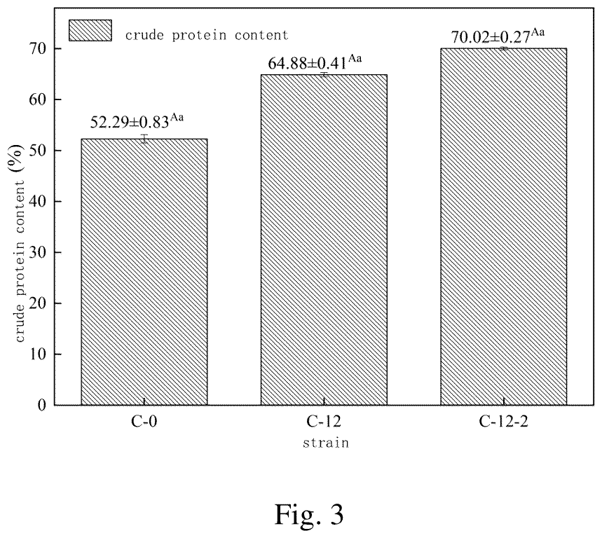

The growth curve measurement for Saccharomyces cerevisiae C-12-2 was performed in three parallel repetitions, and the data results are shown in FIG. 2.

As shown in FIG. 2, the growth trend of Saccharomyces cerevisiae C-12-2 strain can be summarized as follows: from 0 to 4 hours, the strain is in the lag phase; from 4 to 22 hours, the cell count increases exponentially, indicating the strain has entered the logarithmic growth phase; from 22 to 28 hours, the biomass reaches its peak, which corresponds to the stationary phase. During the logarithmic growth phase, the strain has sufficient nutrients, a fast growth rate, active metabolism, and relatively stable cellular morphology and physiological characteristics. The strain is also more sensitive to changes in physical and chemical factors, making it prone to mutations. Therefore, cells in this growth phase are typically selected for experimental research. By constructing a growth curve, the metabolic patterns of different strains can be assessed, and optimal inoculation times can be determined. Under the same conditions, the faster the growth rate of the strain, the better its utilization of nutrients, making it more advantageous for practical applications.

### 3. Single-Cell Protein Measurement

The Saccharomyces cerevisiae C-12-2 seed culture was inoculated at a 1% (v/v) inoculum (OD₆₀₀=0.8) into 100 mL/250 mL YPD liquid medium and incubated at 30° C. and 180 rpm for 24 hours. After 24 hours of fermentation, the cell suspension was centrifuged at 4500 rpm for 10 minutes, and the supernatant was discarded. The pellet was washed with deionized water and centrifuged again at 4500 rpm for 10 minutes. The washing step was repeated 3 times. The cells were then placed in a 105° C. oven and dried to constant weight. After cooling, 0.1 g of the sample was accurately weighed, and the crude protein content was determined following the method in the standard “GB/T 6432-2018 Determination of Crude Protein in Feeds by Kjeldahl Method.”

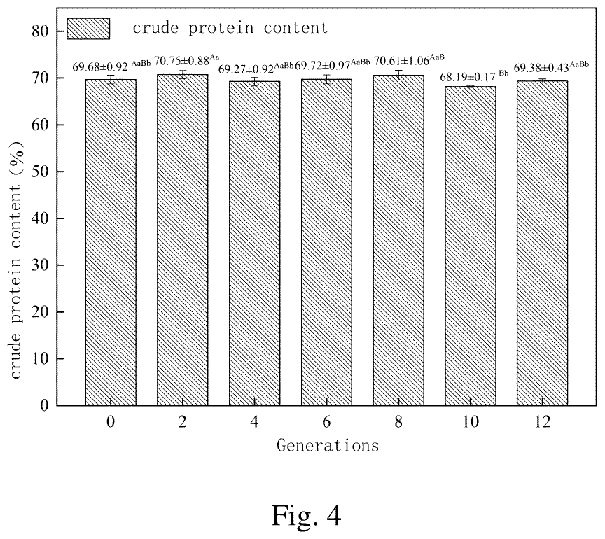

The crude protein content of Saccharomyces cerevisiae C-0, C-12, and Saccharomyces cerevisiae C-12-2 strains was measured in three parallel experiments. The data results are presented as the mean±standard deviation for statistical significance analysis. The crude protein content results for Saccharomyces cerevisiae C-0, C-12, and Saccharomyces cerevisiae C-12-2 strains are shown in FIG. 4.

As shown in FIG. 3, after acid tolerance acclimatization, the single-cell protein content of Saccharomyces cerevisiae C-12-2 reached 70.02%, which is a 33.91% increase compared to the original strain Saccharomyces cerevisiae CGMCC 2.2878 (C-0). Furthermore, Saccharomyces cerevisiae C-12-2 has the ability to grow under low pH conditions, making it suitable as a strain for subsequent fermentation applications.

### 4, Genetic Stability Test of Mutant and Acclimatized Strain

The genetic stability of the mutant and acclimatized Saccharomyces cerevisiae C-12-2 strain was analyzed. The specific method was as follows: The Saccharomyces cerevisiae C-12-2 strain was subjected to continuous passage for 12 generations. The strain from the mutation and acclimatization process was designated as generation 0. The 0th generation culture was inoculated at a 1% (v/v) inoculum into YPD liquid medium with pH 3.5 and incubated at 30° C. and 180 rpm for 12 hours (OD₆₀₀=2) to obtain the 1st generation seed culture. The 1st generation seed culture was then inoculated at a 1% (v/v) inoculum into YPD liquid medium with pH 3.5 and incubated under the same conditions for 12 hours (OD₆₀₀=2) to obtain the 2nd generation seed culture. This process was repeated continuously for 12 generations. Every 2 generations, the crude protein content was measured according to the method in GB/T 6432-2018.

The crude protein content of Saccharomyces cerevisiae C-12-2 strains from generations 0, 2, 4, 6, 8, 10, and 12 was measured in three parallel experiments. The data results are presented as the mean±standard deviation for statistical significance analysis. The crude protein content measurement results for Saccharomyces cerevisiae C-12-2 are shown in FIG. 4.

As shown in FIG. 4, The differences among the various Cyberlindnera jadinii C-12-2 strains are not obvious, indicating that the performance of Saccharomyces cerevisiae C-12-2 in single-cell protein production is stable. The performance stability experiment demonstrates that the crude protein content of Saccharomyces cerevisiae C-12-2, selected by heavy ion mutation, maintains good stability.

In conclusion, this application provides an acid-tolerant Saccharomyces cerevisiae C-12-2 strain, which was developed through mutation selection using ¹²C⁶⁺ heavy ion beams, followed by acid tolerance acclimatization. The resulting strain is not only acid-tolerant and high in protein content, but also has stable performance. This offers a new approach for the selection and application of acid-tolerant, high-protein yeast strains. Furthermore, it lays a theoretical and practical foundation for using Saccharomyces cerevisiae as a production strain for microbial protein and protein feeds.

The above embodiments are preferred implementation examples of this application and should not be used to limit the scope of protection of this application. Any modifications or refinements made within the spirit and design concept of this application, which solve the same technical problems, should be included within the scope of protection of this application.

What is claimed is:

1. A strain of acid-tolerant Cyberlindnera jadinii yeast, wherein the Cyberlindnera jadinii yeast is Cyberlindnera jadinii C-12-2, which was deposited on Mar. 5, 2024, at the Guangdong Microbial Culture Collection Center, with the deposit number GDMCC NO: 64388, and the deposit address is: 5th Floor, Building 59, No. 100 Xianlie Middle Road, Guangzhou, Guangdong, China.
